(19) United States
(12) Patent Application Publication
(10) Pub. No.: US 2024/0157559 A1
(43) Pub. Date: May 16, 2024

(54) REAL-TO-SIMULATION MATCHING OF DEFORMABLE SOFT TISSUE AND OTHER OBJECTS WITH POSITION-BASED DYNAMICS FOR ROBOT CONTROL

(71) Applicant: The Regents of the University of California, Oakland, CA (US)

(72) Inventors: Fei LIU, La Jolla, CA (US); Michael C. YIP, La Jolla, CA (US); Florian RICHTER, La Jolla, CA (US)

(21) Appl. No.: 18/281,472

(22) PCT Filed: Mar. 31, 2022
(86) PCT No.: PCT/US22/22820

§ 371 Date: Sep. 11, 2023

Related U.S. Application Data
(60) Provisional application No. 63/168,499, filed on Mar. 31, 2021.

(51) Int. Cl. B25J 9/16

(52) U.S. Cl. CPC B25J 9/1671; B25J 9/1694

(57) ABSTRACT

A method is provided for generating and updating a simulation of one or more objects from sensory data. The method includes: (i) receiving sensory data; (ii) detecting one or more objects in the sensory data; (iii) initializing both a simulator geometry of the one or more objects in a simulator and simulator parameters used in the simulator; (iv) predicting the simulator geometry using the simulator parameters; (v) computing predicted sensory data from the predicted simulator geometry; (vi) computing a loss between the predicted sensory data and the received sensory data; (vii) updating the simulator geometry and the simulator parameters by minimizing the computed loss; (viii) repeating (i)-(viii) if new sensory data is received; and (ix) providing a simulation of the one or more objects using the updated simulator geometry and the updated simulator parameters.

### CROSS REFERENCE TO RELATED APPLICATION

This application claims the benefit of U.S. Provisional Application Ser. No. 63/168,499, filed. Mar. 21, 2021, the contents of which are incorporated herein by reference.

### BACKGROUND

To successfully navigate in and interact with the 3D world we live in, a 3D geometric understanding is required. For environments with complex, non-rigid objects (e.g. tissue, ropes, liquids), an additional dynamic understanding is required for interaction. As humans, we gain experiences of how to handle dynamic objects. For example, the fluid dynamic laws that govern liquids are implicitly understood when conducting the simple task of pouring a cup of coffee. Data driven approaches attempt to replicate this ability by exploring and interacting with the environment (e.g. Reinforcement Learning) or learning from demonstrations of the task. However, these approaches fail to generalize to tasks outside their training data and do not have an explicit model of the real world.

### SUMMARY

In accordance with one aspect of the subject matter described herein, a method is provided for generating and updating a simulation of one or more objects from sensory data. The method includes: (i) receiving sensory data; (ii) detecting one or more objects in the sensory data; (iii) initializing both a simulator geometry of the one or more objects in a simulator and simulator parameters used in the simulator; (iv) predicting the simulator geometry using the simulator parameters; (v) computing predicted sensory data from the predicted simulator geometry; (vi) computing a loss between the predicted sensory data and the received sensory data; (vii) updating the simulator geometry and the simulator parameters by minimizing the computed loss; (viii) repeating (i)-(viii) if new sensory data is received; and (ix) providing a simulation of the one or more objects using the updated simulator geometry and the updated simulator parameters.

In accordance with another example of the subject matter described herein, a robot manipulates the one or more objects and the method further includes: receiving kinematic information of the robot; receiving robot action information concerning actions performed by the robot manipulating the one or more objects, wherein receiving the sensory data includes receiving sensory data concerning the one or more objects being manipulated by the actions performed by the robot and wherein predicting the simulator geometry also uses the robot action information.

In accordance with another example of the subject matter described herein, minimizing the computed loss includes minimizing the computed loss uses a minimization technique selected from the group consisting of gradient descent, a Levenberg-Marquardt algorithm, a Trust Region Optimization technique, and a Gauss-Newton algorithm.

In accordance with another example of the subject matter described herein, a derivative for the minimization technique is computed using auto-differentiation, finite difference, adjoint method or is analytically derived.

In accordance with another example of the subject matter described herein, receiving robot action includes receiving robot joint angle, velocity and/or torque measurement information.

In accordance with another example of the subject matter described herein, the simulator is a position-based dynamics simulator.

In accordance with another example of the subject matter described herein, the simulator is a rigid body dynamics simulator.

In accordance with another example of the subject matter described herein, the simulator is an articulated rigid body dynamics simulator.

In accordance with another example of the subject matter described herein, the simulator is a smooth particular hydrodynamics simulator.

In accordance with another example of the subject matter described herein, the simulator is a finite element method-based dynamics simulator.

In accordance with another example of the subject matter described herein, the simulator is a projective dynamics simulator.

In accordance with another example of the subject matter described herein, the simulator is an energy projection-based dynamics simulator.

In accordance with another example of the subject matter described herein, the sensory data includes image data, CT/MRI scans, ultrasound, depth image data, and/or point cloud data

In accordance with another example of the subject matter described herein, the sensory data is expanded over a predetermined time window encompassing multiple iterations of simulation time steps.

In accordance with another example of the subject matter described herein, the one or more objects includes at least one deformable object.

In accordance with another example of the subject matter described herein, the one or more objects includes at least one rigid body.

In accordance with another example of the subject matter described herein, the one or more objects includes at least one articulated rigid body.

In accordance with another example of the subject matter described herein, the one or more objects includes at least one deformable linear object.

In accordance with another example of the subject matter described herein, the at least one deformable linear object is selected from the group consisting of rope, suture thread and tendons.

In accordance with another example of the subject matter described herein, the one or more objects includes at least one liquid.

In accordance with another example of the subject matter described herein, the one or more objects includes at least two different objects that interact with one another.

In accordance with another example of the subject matter described herein, the method further includes manipulating the one or more objects in accordance with the simulation so that a physical geometry of the one or more objects aligns with a goal geometry.

In accordance with another example of the subject matter described herein, the simulation is updated during manipulation of the one or more objects to provide closed-loop control.

In accordance with another example of the subject matter described herein, the simulation is used to provide open-loop control.

In accordance with another example of the subject matter described herein, the method further includes computing a control loss between the goal geometry and the simulator geometry and minimizing the control loss to compute a sequence of robot actions that are used to manipulate the one or more objects.

In accordance with another example of the subject matter described herein, the method further includes executing the sequence of robot actions to manipulate the one or more objects such that the physical geometry of the one or more objects aligns with the goal geometry.

In accordance with another example of the subject matter described herein, minimizing the control loss uses a minimization technique selected from the group consisting of gradient descent, a Levenberg-Marquardt algorithm, a Trust Region Optimization technique, and a Gauss-Newton algorithm.

In accordance with another example of the subject matter described herein, a derivative for the minimization technique is computed using auto-differentiation, finite difference, adjoint method or is analytically derived.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 is a flowchart of an illustrative method for generating and continuously updating a simulation of object(s) of interest from sensory (e.g., image) data while being manipulated by a robot.

FIG. 2 is a flowchart of an illustrative method performed by a controller for instructing a robot to manipulate object(s) of interest such that the physical geometry of the object(s) of interest aligns with a goal geometry, where the controller uses a simulation of the object(s) of interest that is obtained from the method of FIG. 1.

FIG. 3 shows an outline of a single timestep in a process for predicting the simulator geometry that is performed by a simulator algorithm.

FIG. 4 illustrates the distance constraint used when modeling deformable objects.

FIG. 5 illustrates the volume preservation constraint used when modeling deformable objects.

FIG. 6 illustrates the shape matching constraint used when modeling deformable objects.

FIG. 7 illustrates the joint positional constraint used when modeling deformable objects.

FIG. 8 illustrates the joint angular constraint used when modeling articulated rigid objects.

FIG. 9 shows the discretization of a deformable linear object using a sequence of particles.

FIG. 10 is a flowchart of the real-to-sim matching process applied to the manipulation of chicken skin.

FIG. 11 shows a robot manipulating deformable tissue, where the top row of images shows the actual sensory data of the tissue obtained from a camera and the bottom row of images show the simulation of the deformable tissue.

### DETAILED DESCRIPTION

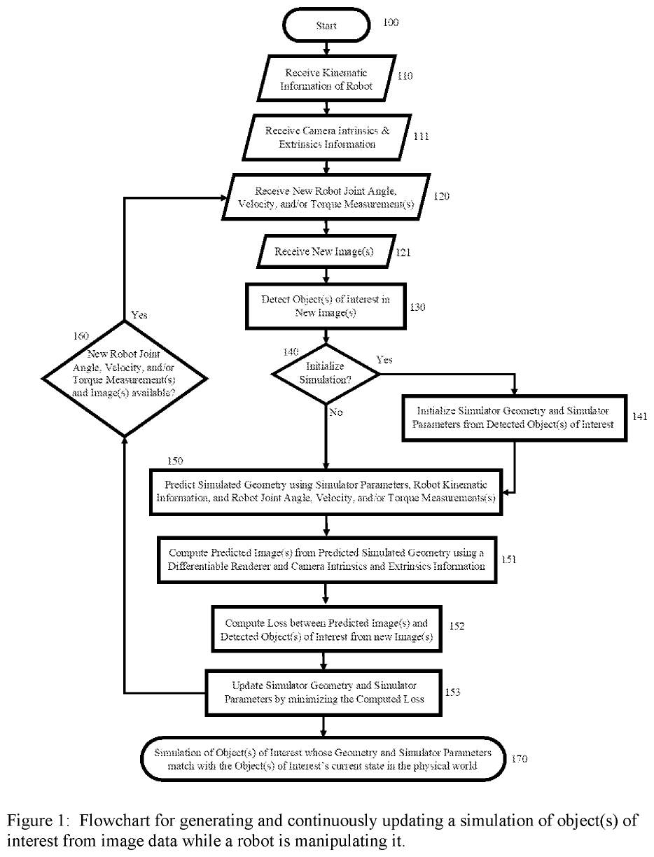

As explained in more detail below, a simulation of one or more objects of interest is created and updated according to their current state in the physical world. The process of creating and updating this simulation is referred to herein as real-to-sim modeling. Real-to-sim provides an explicit model of the real world that generalizes well since it continuously matches a simulation to the real world using sensory data (e.g., image data, CT/MRI scans, ultrasound, depth images, and/or point cloud data). The real-to-sim matching process will be described below in connection with the flowchart of FIG. 1.

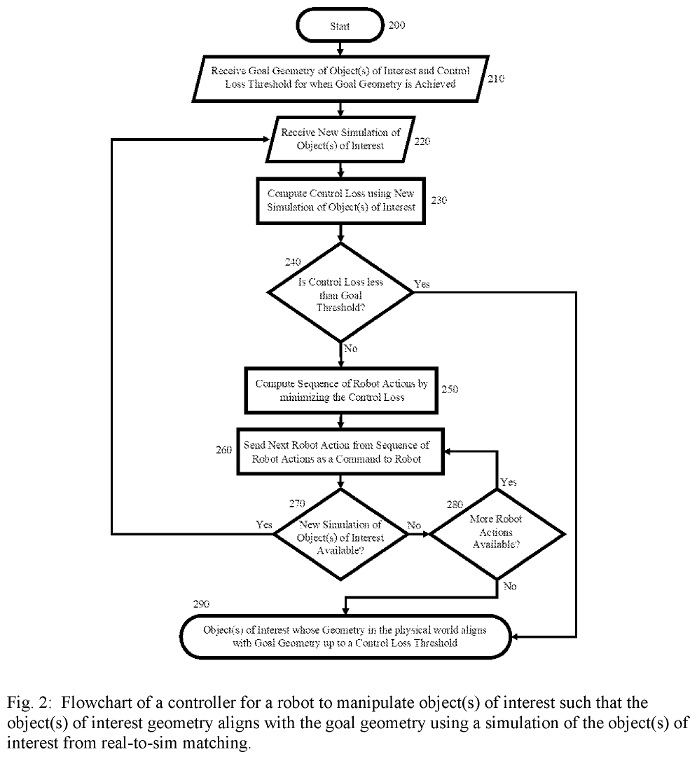

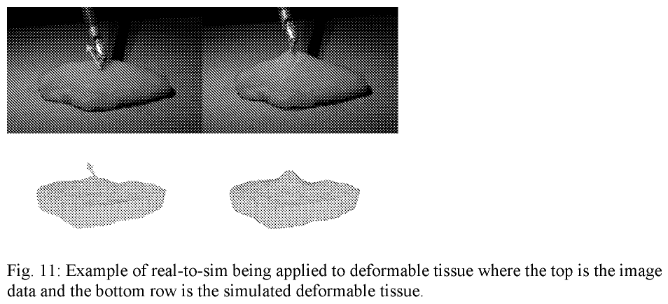

While in principle any simulator can be used for real-to-sim matching, a set of illustrative models is presented below when discussing real-to-sim modelling. These models are based on a Position Based Dynamics (PBD) simulator described in Müller, Matthias, et al., “Position based dynamics,” Journal of Visual Communication and Image Representation vol. 18 no. 2 pp. 109-118, 2007, which is hereby incorporated by reference in its entirety. These models are used because of their stability when taking large time-steps. The models are used to simulate various mediums in PBD such as deformable objects, ropes, and rigid bodies. Since the simulation is being updated to match the current physical world, a controller or other downstream application can leverage the simulation to predict how the object(s) of interest will behave so that they can be interacted with. For instance, FIG. 11 shows an example in which a robot is manipulating deformable tissue, where the top row of images shows the actual sensory data of the tissue obtained from a camera and the bottom row of images show the simulation of the deformable tissue. Real-to-sim control will be described below in connection with the flowchart of FIG. 2.

### Real-to-Sim Matching

In this section, we detail embodiments of real-to-sim matching for an object(s) of interest. A flowchart of the real-to-sim matching process is shown in FIG. 1. The simulator is denoted as a function ƒ(⋅) that takes the simulation from time-step t to t+1:

pₜ₊₁, p_{\dot{t}+1}=ƒ(pₜ, \dot{p}ₜ, aₜ|s)

where pₜ is the positional information of the simulator (i.e. geometry), \dot{p}ₜ is pₜ's corresponding velocity, aₜ is the action information of a manipulation being applied on the object(s) of interest (e.g. joint angles for robot interaction) and s are the simulator parameters (e.g. stiffness and direction of gravity). Simulators such as rigid body dynamics simulator (e.g. Bullet) or smooth particular hydrodynamics could be used for ƒ(⋅). In the real-to-sim modelling section presented below, we cover a specific usage of a PBD simulator for ƒ(⋅). The goal of real-to-sim is to continuously solve for the object(s) of interest geometry and simulator parameters (pₜ, \dot{p}ₜ, s) from sensory data of the real world at every timestep.

Solving for (pₜ, \dot{p}ₜ, s) is done at every timestep by minimizing the error between the simulator's predicted sensory data and the measured sensory data (e.g. matching a rendered image of the simulation and an image from the physical world). This optimization problem can be written explicitly as a loss, [symbol] (⋅,⋅) (e.g. mean square error), between the sensory data, zₜ₊₁, and the simulator's predicted sensory data, h(⋅), as:

$${arg min}_{{p}_{t} , \dot{{p}_{t}} , s} ℒ ( {z}_{t + 1} , h ( f ( {p}_{t} , \dot{{p}_{t}} , {a}_{t} \vert s ) ) )$$

This loss is minimized every time new sensory data is received, hence keeping the simulator up to date with the physical world. While any optimization techniques (e.g. Levenberg-Marquardt algorithm, Trust Region Optimization technique, and Gauss-Newton algorithm) can be used to minimize the loss, an approach utilizing gradient descent is illustrated herein. Gradient descent iteratively minimizes loss by computing the following for (pₜ, \dot{p}ₜ, s):

$${p}_{t}^{( i + 1 )} = {p}_{t}^{( i )} - {α}_{p} \frac{\partial ℒ}{\partial h} \cdot \frac{\partial h}{\partial f} \cdot \frac{\partial f}{\partial {p}_{t}} \\ {\dot{p}}_{t}^{( i + 1 )} = {\dot{p}}_{t}^{( i )} - {α}_{\dot{p}} \frac{\partial ℒ}{\partial h} \cdot \frac{\partial h}{\partial f} \cdot \frac{\partial f}{\partial {p}_{t}} \\ {s}^{( i + 1 )} = {s}^{( i )} - {α}_{s} \frac{\partial ℒ}{\partial h} \cdot \frac{\partial h}{\partial f} \cdot \frac{\partial f}{\partial s}$$

where i is the current gradient step and αₚ, α_{\dot{p}}, αₛ are the gradient step sizes. The gradient

$$\frac{\partial h}{\partial f}$$

can be computed for image sensory data using, for example, differentiable renderers such as Christoph Lassner and Michael Zollhofer, “Pulsar: Efficient sphere-based neural rendering,” Proceedings of the IEEE/CVF Conference on Computer Vision and Pattern Recognition, pp. 1440-1449, 2021. We cover simulation techniques such that the simulator gradients,

$$\frac{\partial f}{\partial {p}_{t}} , \frac{\partial f}{\partial {\dot{p}}_{t}} , \frac{\partial f}{\partial s} ,$$

can be computed in the real-to-sim modelling section presented below. Other techniques to compute the derivatives include auto-differentiation, finite difference, adjoint method or analytically derived.

FIG. 1 is a flowchart for real-to-sim matching in which a simulation of object(s) of interest is generated and continuously updated from image sensory data while a robot is manipulating it. The method begins at step 100 and proceeds to steps 110 and 111, which respectively provide the kinematic information (i.e. pose information for a robot) and camera intrinsics and extrinsics. New robot action data (e.g. joint angles, velocity, and torques), aₜ, and image data, zₜ₊₁, is received in steps 120 and 121, respectively. From the image data, the object(s) of interest is detected in step 130, which can be performed using, for example, segmentation techniques such as K. He, et. al., “Mask r-cnn,” Proceedings of the IEEE/CVF Conference on Computer Vision and Pattern Recognition, pp. 2961-2969, 2017. From the first image detections of the object(s) of interest, the simulator parameters are initialized in step 141. This can be done by setting the geometry position, pₜ, to the inverse projection of the detected object(s) of interests in the image data, the geometry's velocity, \dot{p}ₜ, to 0, and simulator parameters s to a default value according to the simulator being used. The simulator is then predicted to the image data time-step with the simulator geometry, simulator parameters, and robot data in step 150. Predicted image(s) that are to be matched with the image detection(s) are computed from the predicted simulator geometry in step 151 using a renderer and the camera intrinsics and extrinsics.

A loss between the image detections and the predicted simulated geometry is computed in step 152. By minimizing the loss, the simulator geometry, pₜ and \dot{p}ₜ, and simulator parameters, s, are updated at step 153. In this example, gradient descent is used to minimize the loss so a differentiable renderer is applied to compute

$$\frac{\partial h}{\partial f}$$

and a simulator discussed in the real-to-sim modelling section is used to compute

$$\frac{\partial f}{\partial {p}_{t}} , \frac{\partial f}{\partial {\dot{p}}_{t}} , \frac{\partial f}{\partial s} .$$

The real-to-sim matching is repeatedly done as new image(s) and robot data is received. As shown in step 160, the entire real-to-sim matching process is repeated (i.e. steps 120-153) every time new robot action data and image data is received. Asynchronous to the repeated process, a simulation of the object(s) of interest whose geometry and simulator parameters match with the object(s) of interest's current state in the physical world is provided as an output of the method in step 170.

In order to stabilize the simulator parameters, the loss can be extended over a window of sensory data. To represent this, the loss function can be re-written as follows:

$${arg min}_{{p}_{t - w : t} , {\dot{p}}_{t - w : t} , s} {\sum }_{k = t - w}^{t} {β}_{k} ℒ ( {z}_{k + 1} , h ( f ( {p}_{k} , {\dot{p}}_{k} , {a}_{k} \vert s ) ) )$$

where w is the window size and βₖ are the weightings for each timestep. The weighting can be uniformly set

$$( {β}_{k} = \frac{1}{w} )$$

or adjusted such that the most recent sensory data has more weight than sensory older data.

### Real-to-Sim Modelling

In this section we detail a specific illustrative implementation for the simulator, ƒ(⋅), which is based on PBDs. PBDs has been a popular approach in recent years for fast simulation of particle-based dynamics. PBD is different from traditional force-based methods, such as Euler-Lagrange formulation. Many geometrical constraints can be applied to solve the integration and prediction of dynamical states. A detailed review for various models of common objects such as cloth, deformable bodies, and fluid can be found in the aforementioned Müller, Matthias, et al. reference. Of course, as previously mentioned, a wide variety of alternative simulators may be used instead of PBD.

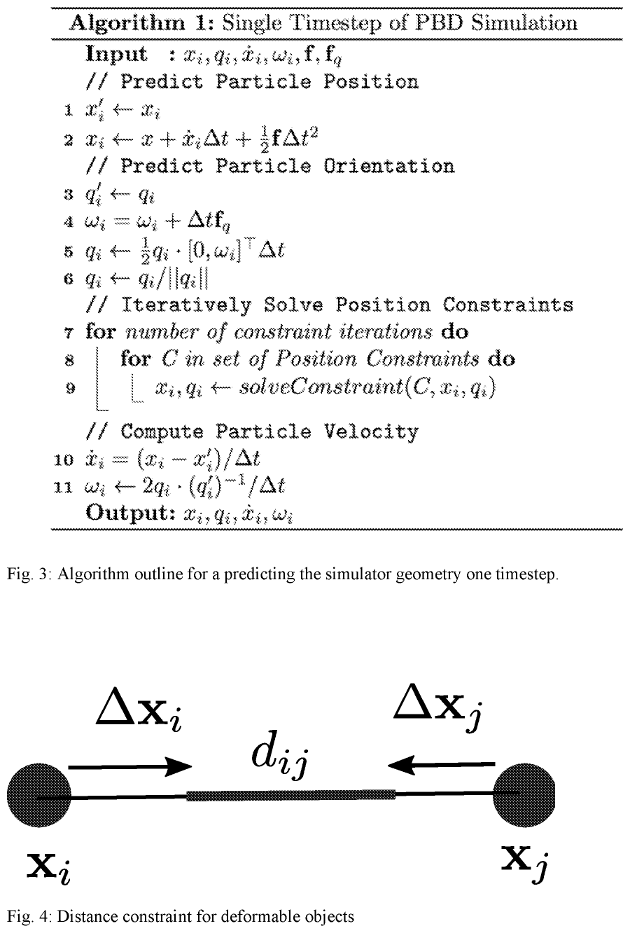

An outline of the algorithm for a general PBD simulator is shown in FIG. 3. PBDs are particle-based dynamics, so in the coming sub-sections and in FIG. 3, we define xᵢ as the i-th particle in the simulator, \dot{x}ᵢ as the i-th particle velocity, and the simulator geometry are simply the set of particles and their respective velocity. For some medium, e.g., rigid bodies, the simulator geometry is extended with orientation in the form of a quaternion, qᵢ, and angular velocity, ωᵢ. In line 2 of FIG. 3, the particles' positions are predicted with Newton's equation of motion where f is the applied acceleration (e.g. gravity or robot action). When the particles include orientation, the particle orientation is predicted with Newton's equation of motion in lines 4-6 of FIG. 3, where f_{q} is the applied angular acceleration (e.g. robot action). To simulate the different mediums with PBDs (e.g. deformable objects, rigid bodies, and ropes) only position constraints need to be defined in order to fully define a model of that medium, and they are iteratively solved in line 7-9 of FIG. 3. The constraints can be solved using the Gauss-Newton algorithm. Finally, the velocity is estimated in lines 10 and 11 of FIG. 3 for linear and angular velocities respectively. In the coming subsections, we define constraints for different mediums, hence completely defining models for the real-to-sim technique described herein. We make the PBD simulator differentiable with respect to simulator geometry and simulator parameters:

$$\frac{\partial f}{\partial {p}_{t}} , \frac{\partial f}{\partial {\dot{p}}_{t}} , \frac{\partial f}{\partial s} .$$

The gradients can be computed using auto-differentiable frameworks such as PyTorch, TensorFlow, finite difference, or adjoint methods.

Robot actions aₜ are incorporated in the simulation as an applied acceleration f in line 1 of FIG. 3. The applied accelerations can be computed from joint angle, velocity, and torque measurements and the robot kinematic information. Another approach is to apply it as a position constraint as done in J. Huang et al., “Model-predictive control of blood suction for surgical hemostasis using differentiable fluid simulations,” IEEE International Conference on Robotics and Automation, pp. 12380-12386, 2021. Similar to simulator geometry and simulator parameters, the PBD simulator can be made differentiable with respect to robot action,

$$\frac{\partial f}{\partial {a}_{t}} ,$$

so a robot action can be optimized, as done in the real-to-sim control section presented below. The gradients can be computed using auto-differentiable frameworks such as PyTorch, TensorFlow, finite difference, or adjoint methods.

Deformable Objects: Different from the traditional Euler-Lagrangian dynamics modeling approach, PBDs discretizes deformable objects as particles with constraint relationships. The geometric constraints are defined as functions of positional information of particles. Thus, the deformable materials are identified not by their physical parameters but through constraint equations which define particle positions and position-derivatives. Here, we introduce several typical geometrical constraints for deformable objects.

a) Distance Constraint: the distance constraint is to preserve the distance between the adjacent pair of particles to the rest shape. For each pair of neighboring particle pairs indicated by indices {i,j}, we have the following equation to be solved,

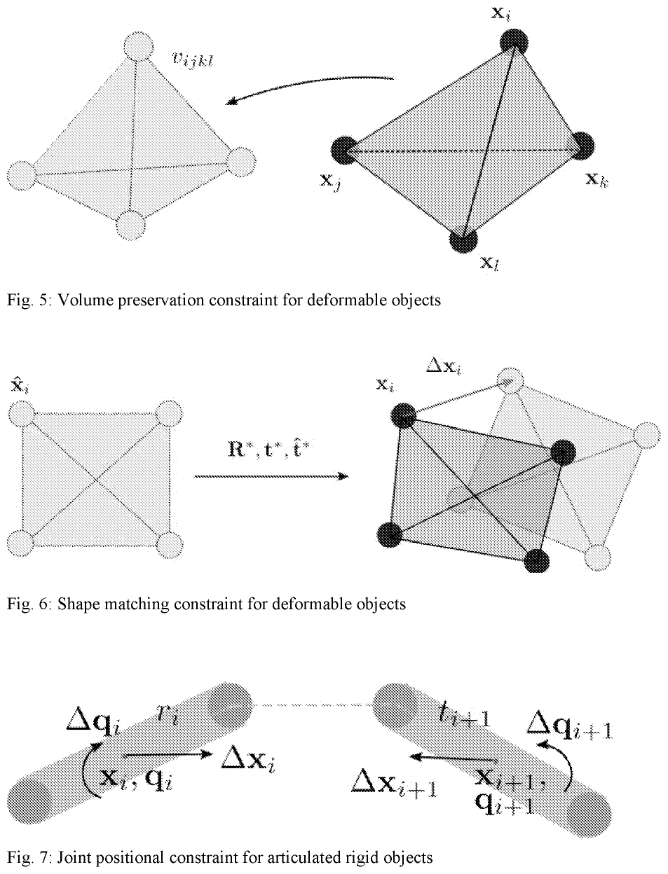

C_{dis}(xᵢ, xⱼ)=∥xᵢ−xⱼ∥−dᵢⱼ
where dᵢⱼ is the distance between particle i and j in rest shape, as shown in FIG. 4.
   b) Volume Preservation: the volume of a tetrahedral, represented by the four particles formulate a tetrahedral mesh, that is

$${C}_{vol} ( {x}_{i} , {x}_{j} , {x}_{k} , {x}_{l} ) = \frac{1}{6} ( ‖ {x}_{j} - {x}_{i} ‖ \times ‖ {x}_{k} - {x}_{i} ‖ \cdot ‖ {x}_{l} - {x}_{i} ‖ ) - {v}_{ijkl}$$

where Vᵢⱼₖᵢ is the rest volume for the tetrahedron, as shown in FIG. 5.
c) Shape Matching: shape matching is a geometrically motivated approach of simulating deformable objects preserve rigidity. The basic idea is to separate the particles into several local cluster regions and then, to find the best transformation that matches the set of particle positions (within the same cluster) before and after deformation, denoted by \hat{x}ᵢ and xᵢ, respectively. Note that \hat{x}ᵢ is the position of the particle at t=0. The corresponding rotation matrix R and the translational vector \hat{t}, t of each cluster are determined by minimizing the total error,

$$arg {min}_{R , \hat{t} , t} {C}_{shape} = {\sum }_{i}^{n} {‖ R ( {\hat{x}}_{i} - \hat{t} ) + t - {x}_{i} ‖}_{2}^{2}$$

where n represents the number of particles in the corresponding cluster, as shown in FIG. 6.

The simulator parameters when simulating deformable objects with PBDs and using these constraints are dᵢⱼ, vᵢⱼₖₗ, \hat{x}ᵢ.

Rigid Bodies: Different from the above deformable object, we need to define a rigid body which can both translate and rotate in space. The particle representation per link (i.e., rigid body) is extended with orientation information, qᵢ, to model joint kinematic constraints for robot manipulators. It should be noted that each link of the articulated robot connected by joints can be represented as a single particle with both positional and angular constraints.
Positional Constraints: for each pair of two connected links, each represented by particles located at their respective centers of mass (COM), i.e., x, xᵢ₊₁, with the quaternion representing orientation denoted as qᵢ, qᵢ₊₁ respectively. The positional constraint aims to solve the correction terms at their center of masses that ensure that the particle distances relative to the hinge are constant,

Cⱼₒᵢₙₜₚₒₛ(xᵢ, xᵢ₊₁, qᵢ, qᵢ₊₁)=[xᵢ₊₁+R(qᵢ₊₁)tᵢ₊₁]−[xᵢ+R(qᵢ)rᵢ]

where tᵢ₊₁ and rᵢ are local position vector to the hinge relative to the COM and R(⋅) are the rotation matrix represented by rotation vector represented by quaternions, from local frame to world frame. Detailed illustrations can be found in FIG. 7.
Angular Constraints: Hinge joint angular constraint aims at aligning the rotational axes of two connected links, i.e., i and i+1. They are attached to the same hinge joint. Let vᵢ and uᵢ₊₁ be the normalized rotational axis vector in local frame for link i and i+1 respectively. Then, a generalized angular constraint should be satisfied by,

C_{jointang}(qᵢ, qᵢ₊₁)=[R(qᵢ)vᵢ]×[R(qᵢ₊₁)uᵢ₊₁]

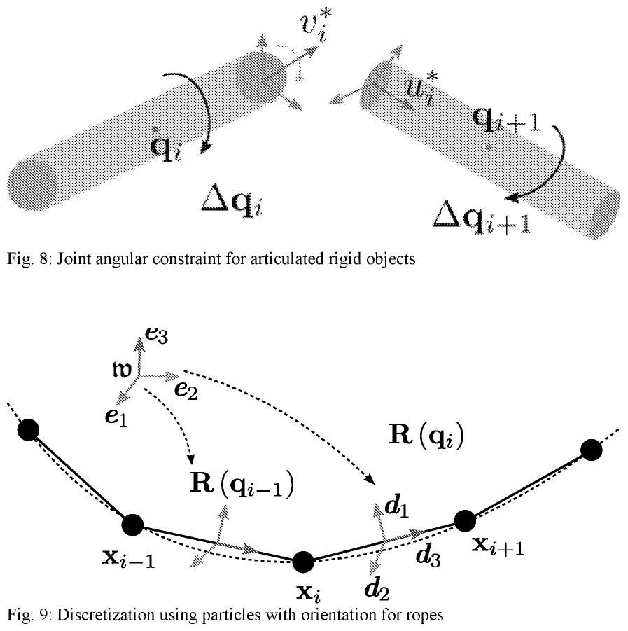

This constraint is shown in FIG. 8 with the same shared symbols representations as above.

The simulator parameters when simulating rigid bodies with PBDs and using these constraints are tᵢ, rᵢ, vᵢ, uᵢ.

Ropes: Ropes or other deformable linear objects (e.g., rope, suture thread, tendons) can be discretized into a sequence of particles as shown in FIG. 9. The particle positions are represented with Cartesian coordinates xᵢ. Meanwhile, quaternions qᵢ are used to describe orientations in-between adjacent particles. They will be used to solve the bending and twist deformation of the rope shapes with following constraints.
Shear and Stretch Constraints: According to Cosserat theory, the shear and stretch measures the deformation regarding the tangent direction of the rope-like object. Therefore, the stretched or compressed length should be constrained relative its rest pose, which indicates in-extensible elasticity. Simultaneously, the normal direction (the rotated e₃ from world frame as shown in FIG. 9) for each cross-section should be parallel to the tangent direction of object's centerline. It measures the shear strain with respect to non-deformed states. Thus, for each pair of neighboring particles, the shear-stretch deformation can be integrated into a generalized constraint as,

$${C}_{shear} ( {x}_{i} , {x}_{i + 1} , {q}_{i} ) = \frac{{x}_{i + 1} - {x}_{i}}{‖ {x}_{i + 1} - {x}_{i} ‖} - R ( {q}_{i} ) {e}_{3}$$

Bend and Twist Constraint: In differential geometry, the Darboux vector Ω is used to parameterize strain deformation with respect to frame rotation. According to Cosserat theory, the Darboux vector can be expressed as a quaternion by measuring the rod's twist in the tangent direction. The difference between the current and resting configuration, denoted as \hat{q}ᵢ, should be evaluated. Thus, the bend and twist constraint can be computed for each pair of two adjacent quaternions as,

C_{bend}(qᵢ, qᵢ₊₁)=Ω−ξΩ̅=Im(qᵢ*·qᵢ₊₁−\hat{q}ᵢ·\hat{q}ᵢ₊₁)

ξ=sign(Ω+Ω̅)

where Im(*) is the imaginary part of the quaternion, and (⋅)* is the corresponding conjugate quaternion.

The simulator parameters when simulating ropes with PBDs and using these constraints are e₃, Ω, \hat{q}ᵢ.

In some embodiments two or more different object(s) of interest can be modelled, where in some cases various combinations of the objects described above may be modelled together while they are interacting with one another or where there is otherwise a coupling between them. As an illustration, a liquid being poured into a rigid body container where the liquid takes the shape of the container represents an example of two different objects interacting with one another. The tensioning of chicken tissue with a robotic arm, which is discussed below, is an example of two different objects that are coupled to one another. In this example one of the objects is a rigid body and the other object is a deformable object.

### Example of Real-to-Sim Matching for Chicken Skin Manipulation

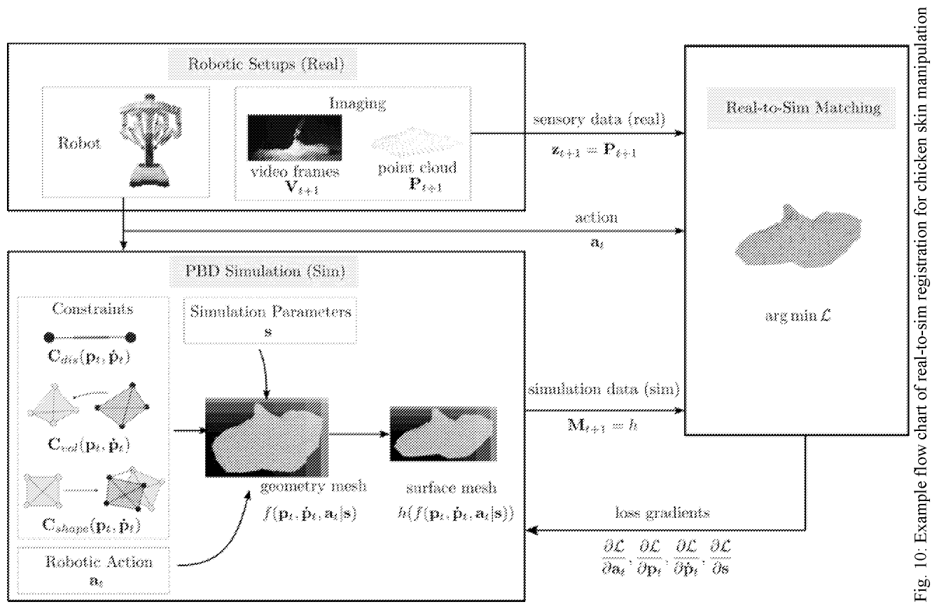

In this section, we present an example of a real-to-sim matching method applied to a chicken skin manipulation scenario by a surgical robot such as shown in FIG. 11. FIG. 10 is a flowchart of the real-to-sim matching. In FIG. 10, the “real” label (i.e., the physical world) denotes a surgical robot and an imaging component (e.g. endoscopic camera, ultrasound, CT/MRI Scanners). In this example, the imaging component provides sensory data, such as videos, Vₜ₊₁, and point cloud data, Pₜ₊₁. The “sim” label in FIG. 10 denotes the PBD simulator that models the chicken skin as a deformable object using the distance, volume and shape matching constraints (Cₛₕₐₚₑ, Cᵥₒₗ, C_{dis}). The simulator geometry position, xᵢ, is initialized using the first point cloud data, P₀, and the simulator geometry velocity, \dot{x}ᵢ, is initialized to 0. The simulator parameters s=(dᵢⱼ, Vᵢⱼₖₗ, \hat{x}ᵢ) are computed using the initial spacing between particle pairs, computed using the initial volume between particle pairs, and set to the initial particle geometry positions respectively. The surgical robot actions, aₜ, is an applied force that is computed from joint measurements and kinematic information from the surgical robot. Finally, the surface mesh, Mₜ₊₁32 h(ƒ(xₜ, \dot{x}ₜ, aₜ|s)), is extracted from the entire geometry mesh represented by the simulator geometry position. The point cloud data is used as sensory data, Pₜ₊₁=zₜ₊₁, at every timestep to compute the real-to-sim matching loss and update the simulator geometry and simulator parameters by minimizing the computed loss. Finally, the updated simulator geometry and simulator parameters with the PBD simulator represents the current state of the “real” (i.e., the physical world) chicken skin as it is being manipulated and stretched.

### Real-to-Sim Control

Since real-to-sim explicitly models object(s) of interest through real-to-sim matching, the simulation of the object(s) of interest can be used to predict how the object(s) of interest will behave with respect to robot actions. This prediction can be utilized for control of the object(s) of interest. In this way, the controller can instruct the robot to manipulate the object(s) of interest so that it conforms to a goal geometry. Let gₜ₊₁, . . . , gₜ₊ₕ be the goal geometry that the controller is to regulate so that the simulator geometry align with the goal geometry for a time horizon of length h. The robot actions are solved for in the simulation to align the simulator geometry with the goal geometry. Since the simulation is matched with the physical world, then executing the computed robots actions on a robot will align the object(s) of interest geometry in the physical world with the goal geometry. Examples of the goals include knot tying and tensioning tissue. The optimal sequence of robot actions, a_{t:t+h}, to be taken is computed by minimizing the following control loss:

$${arg min}_{{a}_{t : t + h}} {\sum }_{k = t}^{t + h} {ℒ}_{c} ( f ( {p}_{k} , {\dot{p}}_{k} , {a}_{k} \vert s ) , {g}_{k + 1} )$$

where [symbol] _{c}(⋅,⋅) is a loss function defined between the predicted and goal geometry of the object(s) of interest (e.g. mean square error). The horizon can also be set to infinity and a discount factor (similar to previous work in Reinforcement Learning) would need to be added to the control loss. The control loss can be minimized using any optimization techniques such as gradient descent, Levenberg-Marquardt algorithm, Trust Region Optimization technique, and Gauss-Newton algorithm. By using a differentiable simulation, such as PBDs described in the real-to-sim modelling section presented above, the control loss can be minimized via gradient descent as follows:

$${a}_{k}^{( i + 1 )} = {a}_{k}^{( i )} - {α}_{a} \frac{\partial ℒ}{\partial f} \cdot \frac{\partial f}{\partial {a}_{k}}$$

for k=t, . . . , t+h where i is the current gradient step and αₐ is the gradient step size. Other techniques to compute the derivative include auto-differentiation, finite difference, adjoint method or analytically derived. The control loss is minimized to re-compute a new sequence of robot actions every time a new simulation from the real-to-sim matching is provided, hence providing closed-loop control. Alternatively, if the simulation is not updated during the execution of robot actions, the control is being done in an open-loop fashion.

A flowchart of the robotic manipulation control process is shown in FIG. 2. First, the goal geometry, gₜ₊₁, . . . , gₜ₊ₕ, and a control loss threshold to define when the goal is achieved are specified and received by the controller in step 210. A new simulation obtained from the real-to-sim matching process described above in connection with FIG. 1 is received in step 220. The control loss is computed in step 230. If the control loss is less than the control loss threshold, then at decision step 240 the physical geometry of the object(s) of interest being controlled is deemed to align with the goal geometry.

On the other hand, if at decision step 240 the control loss is not less than the control threshold, the method proceeds to step 250 where the control loss is minimized to determine the sequence of robot actions, a_{t:t+h}, that will minimize the control loss when applied to the object(s) of interest. The controller instructs the robot to execute the robot actions that have been determined to minimize the control loss. As depicted in steps 260-280, this process is repeated either until there are no more actions or a new simulation from the real-to-matching is received by the controller. Once a new simulation is received from the real-to-sim matching process, the entire loop is repeated. By sending the robot action commands to the robot until no more actions are available at step 280, the method terminates at step 290 where the geometry of the object(s) of interest in the physical world will align with the simulator geometry, which is optimized to align with the goal geometry up to a control loss threshold.

### CONCLUSION

Several aspects of the real-to-sim processes are presented in the foregoing description and illustrated in the accompanying drawing by various blocks, modules, components, steps, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented with a “processing system” that includes one or more processors. Examples of processors include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionalities described throughout this disclosure.

Various embodiments described herein may be described in the general context of method steps or processes, which may be implemented in one embodiment by a computer program product, embodied in, e.g., a non-transitory computer-readable memory, including computer-executable instructions, such as program code, executed by computers in networked environments. A computer-readable memory may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

A computer program product can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

The various embodiments described herein may be implemented in various environments. Such environments and related applications may be specially constructed for performing the various processes and operations according to the disclosed embodiments or they may include a general-purpose computer or computing platform selectively activated or reconfigured by code to provide the necessary functionality. Embodiments described herein may be practiced with various computer system configurations including hand-held devices, tablets, microprocessor systems, microprocessor-based or programmable consumer electronics, minicomputers, mainframe computers and the like. However, the processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the disclosed embodiments, or it may be more convenient to construct a specialized apparatus or system to perform the required methods and techniques. In some cases the environments in which various embodiments described herein are implemented may employ machine-learning and/or artificial intelligence techniques to perform the required methods and techniques.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

1. A method for generating and updating a simulation of one or more objects from sensory data, comprising:
i. receiving sensory data;
ii. detecting one or more objects in the sensory data;
iii. initializing both a simulator geometry of the one or more objects in a simulator and simulator parameters used in the simulator;
iv. predicting the simulator geometry using the simulator parameters;
v. computing predicted sensory data from the predicted simulator geometry;
vi. computing a loss between the predicted sensory data and the received sensory data;
vii. updating the simulator geometry and the simulator parameters by minimizing the computed loss;
viii. repeating (i)-(viii) if new sensory data is received; and
ix. providing a simulation of the one or more objects using the updated simulator geometry and the updated simulator parameters.

2. The method of claim 1, wherein a robot manipulates the one or more objects and further comprising:
receiving kinematic information of the robot;
receiving robot action information concerning actions performed by the robot manipulating the one or more objects; and
wherein receiving the sensory data includes receiving sensory data concerning the one or more objects being manipulated by the actions performed by the robot and wherein predicting the simulator geometry also uses the robot action information.

3. The method of claim 1, wherein minimizing the computed loss includes minimizing the computed loss uses a minimization technique selected from the group consisting of gradient descent, a Levenberg-Marquardt algorithm, a Trust Region Optimization technique, and a Gauss-Newton algorithm.

4. The method of claim 3, wherein a derivative for the minimization technique is computed using auto-differentiation, finite difference, adjoint method or is analytically derived.

5. The method of claim 2, wherein receiving robot action includes receiving robot joint angle, velocity and/or torque measurement information.

6. The method of claim 1, wherein the simulator is a position-based dynamics simulator.

7. The method of claim 1, wherein the simulator is a rigid body dynamics simulator.

8. The method of claim 1, wherein the simulator is an articulated rigid body dynamics simulator.

9. The method of claim 1, wherein the simulator is a smooth particular hydrodynamics simulator.

10. The method of claim 1, wherein the simulator is a finite element method-based dynamics simulator.

11. The method of claim 1, wherein the simulator is a projective dynamics simulator.

12. The method of claim 1, wherein the simulator is an energy projection-based dynamics simulator.

13. The method of claim 1, wherein the sensory data includes image data, CT/MRI scans, ultrasound, depth image data, and/or point cloud data.

14. The method of claim 1, wherein the sensory data is expanded over a predetermined time window encompassing multiple iterations of simulation time steps.

15. The method of claim 1, wherein the one or more objects includes at least one deformable object.

16. The method of claim 1, wherein the one or more objects includes at least one rigid body.

17. The method of claim 1, wherein the one or more objects includes at least one articulated rigid body.

18. The method of claim 1, wherein the one or more objects includes at least one deformable linear object.

19. The method of claim 18, wherein the at least one deformable linear object is selected from the group consisting of rope, suture thread and tendons.

20. The method of claim 1, wherein the one or more objects includes at least one liquid.

21. The method of claim 1, wherein the one or more objects includes at least two different objects that interact with one another.

22. The method of claim 2, further comprising manipulating the one or more objects in accordance with the simulation so that a physical geometry of the one or more objects aligns with a goal geometry.

23. The method of claim 22, wherein the simulation is updated during manipulation of the one or more objects to provide closed-loop control.

24. The method of claim 22, wherein the simulation is used to provide open-loop control.

25. The method of claim 22, further comprising computing a control loss between the goal geometry and the simulator geometry and minimizing the control loss to compute a sequence of robot actions that are used to manipulate the one or more objects.

26. The method of claim 22, further comprising executing the sequence of robot actions to manipulate the one or more objects such that the physical geometry of the one or more objects aligns with the goal geometry.

27. The method of claim 25, wherein minimizing the control loss uses a minimization technique selected from the group consisting of gradient descent, a Levenberg-Marquardt algorithm, a Trust Region Optimization technique, and a Gauss-Newton algorithm.

28. The method of claim 24, wherein a derivative for the minimization technique is computed using auto-differentiation, finite difference, adjoint method or is analytically derived.

29. The method of claim 22, wherein the one or more objects includes at least one deformable object.

30. The method of claim 25, wherein the one or more objects includes at least one deformable object.

31. The method of claim 22, wherein the one or more objects includes at least one rigid body.

32. The method of claim 22, wherein the one or more objects includes at least one articulated rigid body.

33. The method of claim 22, wherein the one or more objects includes at least one deformable linear object.

34. The method of claim 33, wherein the at least one deformable linear object is selected from the group consisting of rope, suture thread and tendons.

35. The method of claim 22, wherein the one or more objects includes at least one liquid.

36. One or more computer-readable storage media containing instructions which, when executed by one or more processors, perform the method of claim 1.
